(19) United States
(12) Patent Application Publication
(10) Pub. No.: US 2018/0164268 A1
(43) Pub. Date: Jun. 14, 2018

(54) HYDROGEN SULFITE DETECTION USING A SPHERE

(71) Applicant: Spectronix Ltd., Sderot (IL)

(72) Inventor: Yossi Ben-Adert, Kibbutz-Zikim (IL)

(21) Appl. No.: 15/709,479

(22) Filed: Sep. 20, 2017

Related U.S. Application Data
(60) Provisional application No. 62/398,233, filed on Sep. 22, 2016.

(51) Int. Cl. G01N 33/00; G01N 21/01; G01N 21/17; G01N 21/47

(52) U.S. Cl. CPC G01N 33/0044; G01N 33/0062; G01N 21/01; G01N 2201/065; G01N 21/4738; G01N 2021/174; G01N 2021/1751; G01N 21/1717

(57) ABSTRACT

A method for sensing Hydrogen Sulfite by a gas sensor, the method may include receiving, by first aperture of a sphere of the gas sensor, a light beam; wherein the sphere further comprises a second aperture and a first gas opening; scattering multiple times, by an interior of the sphere, the light beam to provide a scattered light beam that exits the sphere through the second aperture; receiving, by a gas analyzer of the gas sensor, the scattered light beam from the second aperture; and analyzing the scattered light beam by searching for one or more signs that are indicative of a presence of the Hydrogen Sulfite in the sphere.

### CROSS REFERENCE

This patent application claims priority from U.S. provisional patent Ser. No. 62/398233 filing date 22 Sep. 2017, which is incorporated herein by reference.

### BACKGROUND OF THE INVENTION

Hydrogen Sulfite (H₂S) is a toxic gas.

There is a growing need to detect decreasing concentrations of Hydrogen Sulfite.

### SUMMARY

There may be provided a gas sensor for sensing Hydrogen Sulfite, wherein the gas sensor may include a sphere; and a gas analyzer; wherein the sphere may include a first aperture, a second aperture, and a first gas opening; wherein an interior of the sphere may be configured to scatter multiple times, a light beam that enters the sphere through the first aperture, before a scattered light beam exits the sphere through the second aperture; wherein the gas analyzer may be configured to receive the scattered light beam from the second aperture, and to analyze the scattered light beam by searching for one or more signs that may be indicative of a presence of the Hydrogen Sulfite in the sphere.

The gas sensor may include a light source that may be configured to direct the light beam through the first aperture.

The multiple times exceed three.

The gas sensor may include a gas flow control unit that may be configured to force the Hydrogen Sulfite to flow through the sphere.

The one or more signs may be one or more absorbance patterns that may be indicative of an absorbance of the Hydrogen Sulfite in the scattered light beam.

The light beam may be configured to be scattered N times before exiting from the second aperture, wherein the scattering generates N-1 sets of scattered light beams, wherein the second aperture may be positioned outside a propagation path of each of the N-1 sets of scattered light beams. N is a positive integer that exceeds 1.

The interior of the sphere may be configured to scatter the light beam multiple times thereby defining a folded optical path that may exceed a diameter of the sphere by a factor that may range between 2.5 and five.

The interior of the sphere may be configured to scatter the light beam multiple times thereby defining a folded optical path that may exceed a diameter of the sphere by a factor that may exceed two.

The wavelength of the light beam may range between 180 and 300 nanometer.

The first aperture has a circular shape and the second aperture may be a slit.

The sphere may include a second gas opening.

An imaginary arc passes through the first gas opening, a second gas opening, and the first aperture, wherein the first aperture may be located at the center of the imaginary arc and the first and second gas openings may be located at opposite ends of the imaginary arc.

The gas sensor may include a movement unit that may be configured to move a light source of the gas sensor, the sphere and the gas analyzer.

The gas sensor may include a movement unit that may be configured to introduce movement between the light source and the sphere.

There may be provided a method for sensing Hydrogen Sulfite by a gas sensor, the method may include receiving, by first aperture of a sphere of the gas sensor, a light beam; wherein the sphere may include a second aperture and a first gas opening; scattering multiple times, by an interior of the sphere, the light beam to provide a scattered light beam that exits the sphere through the second aperture; receiving, by a gas analyzer of the gas sensor, the scattered light beam from the second aperture; and analyzing the scattered light beam by searching for one or more signs that may be indicative of a presence of the Hydrogen Sulfite in the sphere.

The method may include directing by a light source of the gas sensor, the light beam through the first aperture.

The multiple times exceed three.

The method may include forcing, by a gas flow control unit, the Hydrogen Sulfite to flow through the sphere.

The one or more signs may be one or more absorbance patterns that may be indicative of an absorbance of the Hydrogen Sulfite in the scattered light beam.

The method may include scattering the light beam, by the sphere, N times before exiting from the second aperture, thereby generating N-1 sets of scattered light beams, wherein the second aperture may be positioned outside a propagation path of each of the N-1 sets of scattered light beams.

The method may include scattering the light multiple times along a folded optical path that may exceed a diameter of the sphere by a factor that may range between 2.5 and five.

The method may include scattering the light multiple times along a folded optical path that may exceed a diameter of the sphere by a factor that may exceed two.

The a wavelength of the light beam may range between 180 and 300 nanometer.

The first aperture has a circular shape and the second aperture may be a slit.

The sphere may include a second gas opening.

An imaginary arc passes through the first gas opening, a second gas opening, and the first aperture, wherein the first aperture may be located at the center of the imaginary arc and the first and second gas openings may be located at opposite ends of the imaginary arc.

The method moving the light source of the gas sensor, the sphere and the gas analyzer.

The method may include introducing movement between the light source and the sphere.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of a gas sensor;

FIG. 2 illustrates an example of a gas sensor;

FIG. 3 illustrates an example of a gas sensor;

FIG. 4 illustrates an example of a gas sensor;

FIG. 5 illustrates an example of a gas sensor;

FIG. 6 illustrates an example of a gas sensor;

FIG. 7 illustrates an example of a method; and

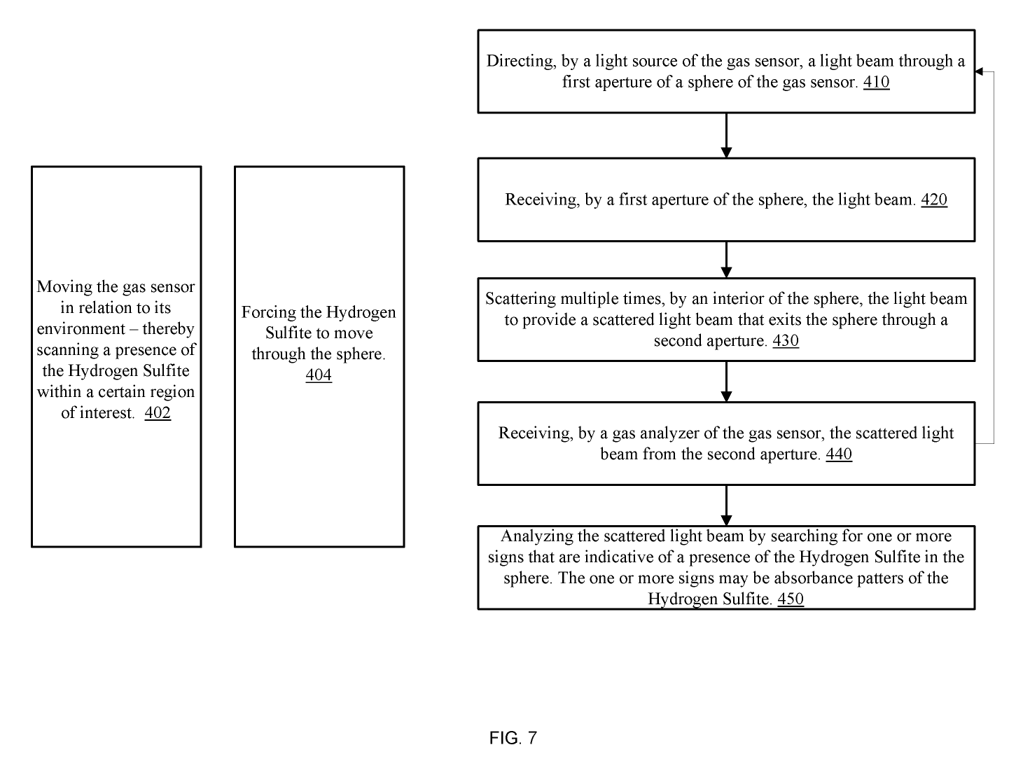

FIG. 8 illustrates an example of a gas sensor.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

The following text may refer to Hydrogen Sulfite but the gas sensor may be used to detect any other gases—especially toxic gases.

According to an embodiment of the invention there is provided a gas sensor that may detect low concentrations of Hydrogen Sulfite—even between 1-5 parts per million (PPM). The gas sensor may also detect higher concentration of Hydrogen Sulfite.

The gas sensor is reliable, highly sensitive, fast and cost effective.

The gas sensor may include a light source, a sphere and a gas analyzer such as a spectrometer for performing broadband absorption spectroscopy in the range of 180-300 nm.

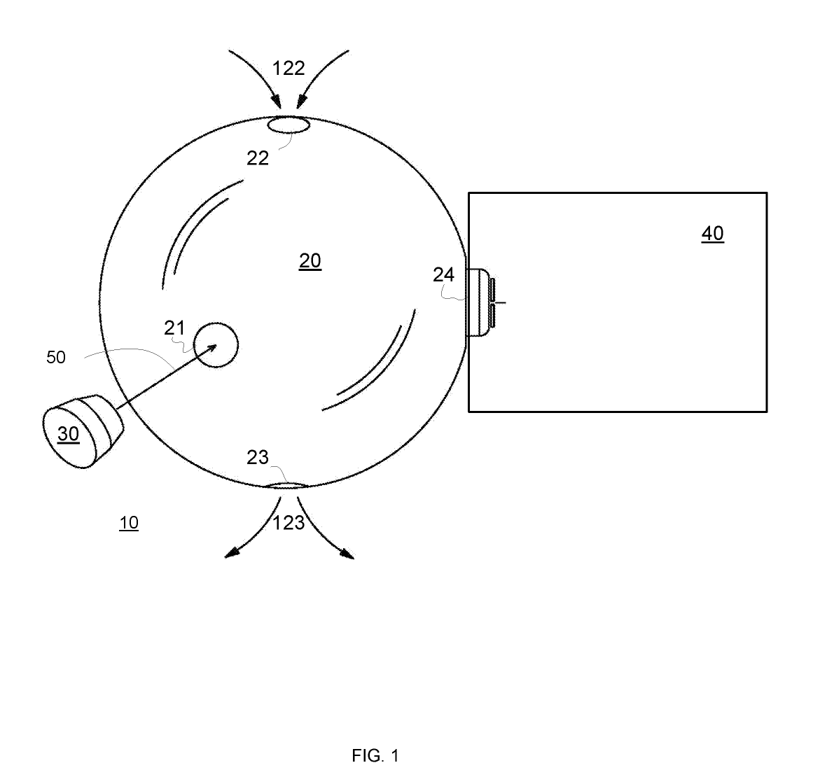

FIG. 1 illustrates an example of gas sensor 10.

Gas sensor 10 includes sphere 20, light source 30 and gas analyzer 40.

In FIG. 1, the sphere 20 is illustrated as including a first aperture 21, a second aperture 24, a first gas opening 22, and a second gas opening 23.

Light beam 50 from light source 30 enters through first aperture 21 of the sphere 20, passes through the Hydrogen Sulfite (if it exists in the sphere), and is scattered from the interior of the sphere to provide one or more scattered light beams that may further interact with the Hydrogen Sulfite and then also be reflected and/or scattered from the interior of the sphere.

This process may be repeated (once or multiple times) until a scattered light beam exits from a second aperture of the sphere—and may be analyzed by the spectrometer.

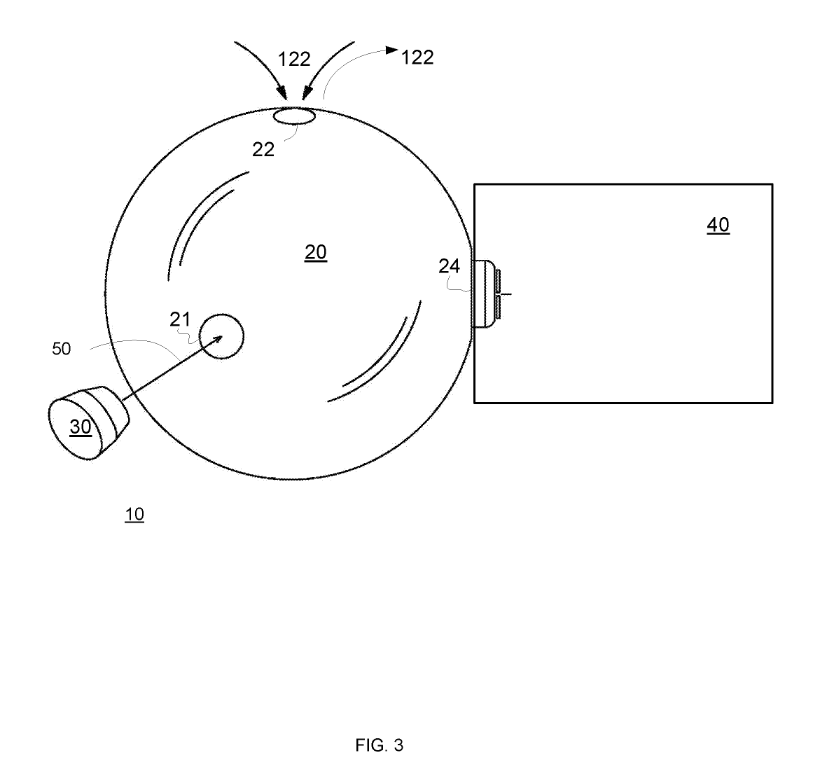

Gas (that may or may not include Hydrogen Sulfite) may enter the sphere through the first gas opening 23 (see arrows 123) and exit the sphere through the first gas opening (see FIG. 3 in which the sphere does not have the second gas opening) or through the second gas opening 23—as shown in FIG. 1.

The gas may be forced to move through the sphere (for example by using air vents)—or may not be forced to move through the sphere.

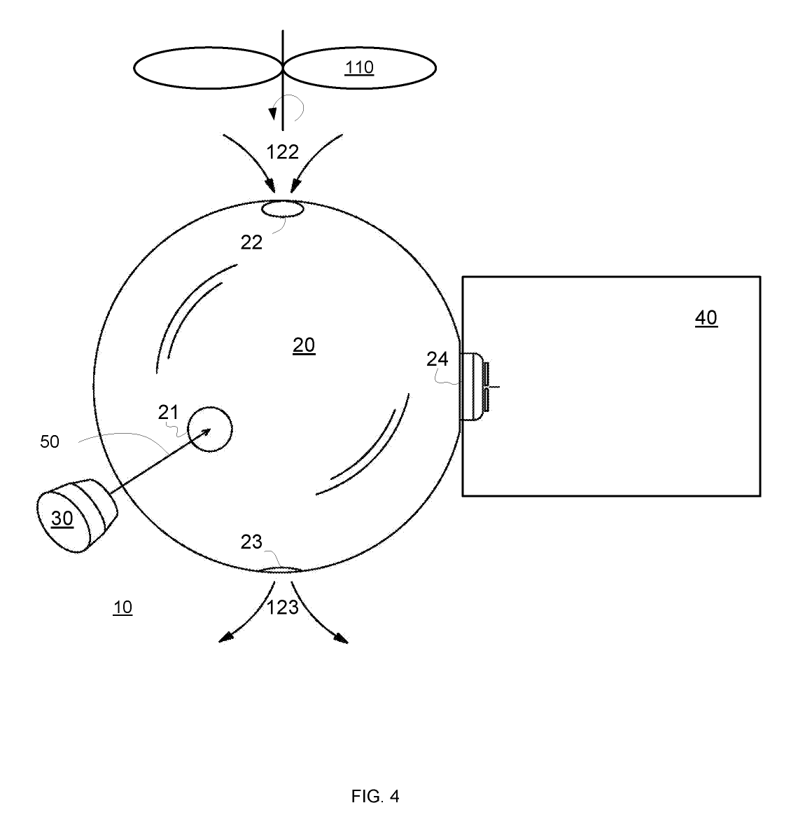

FIG. 4 illustrates a gas flow control unit such as fan 110 that may induce the gas to flow towards the sphere. Any gas flow control unit may be provided and may be located in front of any gas opening of the sphere.

The sphere provides a folded optical path thereby increasing the sensitivity of the gas sensor. The folded optical path enables multiple interactions between light beams (the light beam that entered the sphere any scattered light beams) and the Hydrogen Sulfite, thereby increasing the absorbance of various wavelengths in the Hydrogen Sulfite. The absorbance pattern is detected by the gas analyzer.

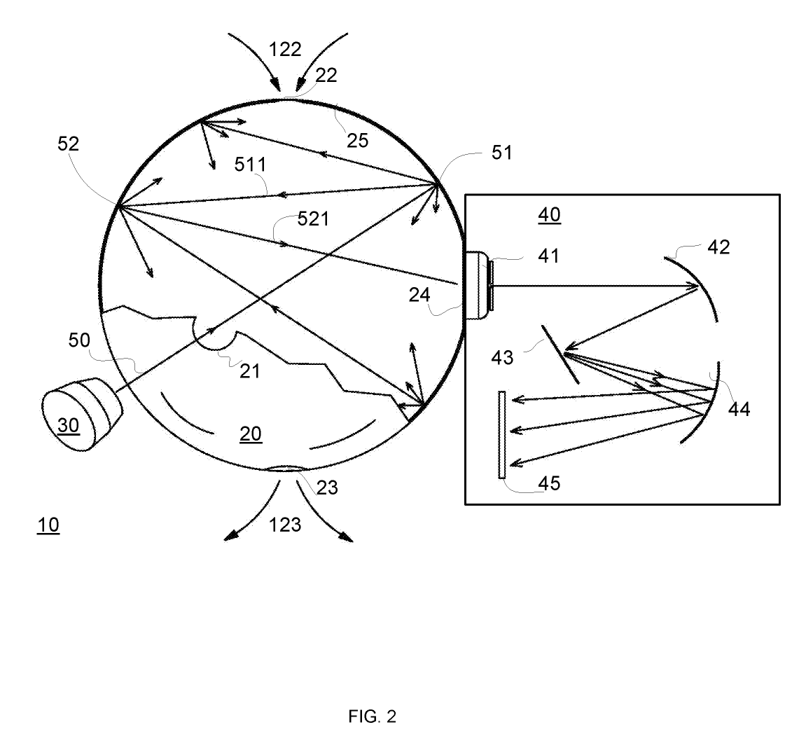

FIG. 2 illustrates a part of the exterior of the sphere and a part of the interior 25 of the sphere.

In FIG. 2:
a. Light beam 50 impinges on the interior 25 of the sphere to provide a first set of scattered light beams 51.
b. Each light beam of the first set may be scattered again to provide one or more second sets of scattered light beams.
c. Scattered light beam 511 of the first set is scattered to provide a second set 52 of scattered light beams.
d. Scattered light beam 521 of the second set 52 exits the sphere through the second aperture 24.

In FIG. 2 the light is scattered twice (N=2) and the length of the folded optical path within the sphere is a sum of:
a. A length of the optical path traveled (within the sphere) by light beam 50.
b. The length of the optical path traveled of light beam 511.
c. The length of the optical path travelled by scattered light beam 521.

In FIG. 2 the light is scattered twice (N=2). It should be noted that N may exceed two.

In FIG. 2 the gas analyzer is a spectrometer that includes a collection port 41 in which a slit is formed, a first focusing mirror 42, a diffraction grating 43, a second focusing mirror 44, and light sensor array 45.

Other spectrometer sand/or other gas analyzers may be provided.

Spectrometer 40 generates a spectrum of the scattered light beam, and based on the spectrum—especially intensity of spectral components—may find one or more signs to the presence of the Hydrogen Sulfite within the sphere. These signs may be absorbance patterns.

The Hydrogen Sulfite has a certain signature that represents certain attenuations in certain spectral components. The spectrometer searches for this signature.

The spectrometer may include or may be coupled to a processor for processing detection signals from the image sensor—and searching for an absorbance pattern that is characteristic of the Hydrogen Sulfite.

In FIG. 2:
a. Scattered light beam 521 propagates through second aperture 24, through the slit of the collection port 41 and impinges on first focusing mirror 42.
b. First focusing mirror 42 focuses the scattered light beam onto diffraction grating 43.
c. Diffraction grating 42 directs different spectral components of the scattered light beam onto different locations of second focusing mirror 44.
d. Second focusing mirror 44 directs the spectral components that impinge on different locations to different light sensors of sensor array 45.
e. Different light sensors of the sensor array 45 sense the different spectral components to provide a spectrum of the scattered light beam.

Non-limiting technical parameters of the sensor may include:
a. Folded optical path (aggregate path passed by light from entering the first aperture and exiting through the second aperture) that ranges between 50 and 100 centimeters.
b. Wavelength of light beam 180-300 nm.
c. Sphere diameter 200 mm.
d. Integration sphere coating reflect 92% Lambertian distribution.
e. Light source may be a Xenon source such as Excelitas RSL-2100.
f. Output port equivalent to fiber 600 um core having a numerical aperture (NA) of 0.16.
g. Spectrometer is Ibsen Freedom UC 190-435 nm OEM Spectrometer.

The gas sensor can be mounted at a fixed position, can be moved in order to scan an area, and the like.

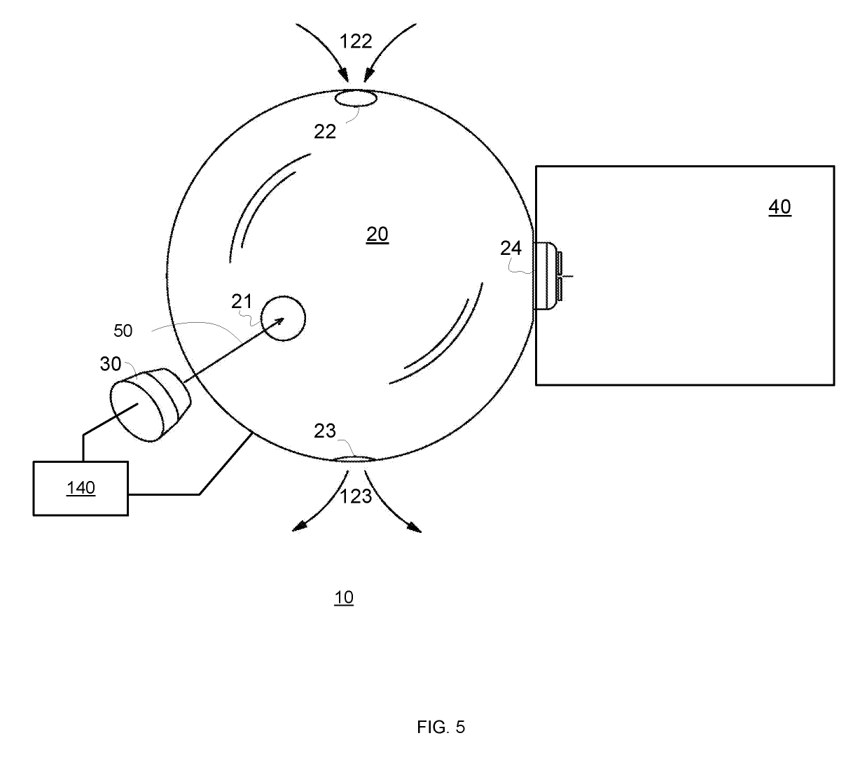

FIG. 5 illustrates a movement unit (such as a mechanical stage) 140 that may move the gas sensor.

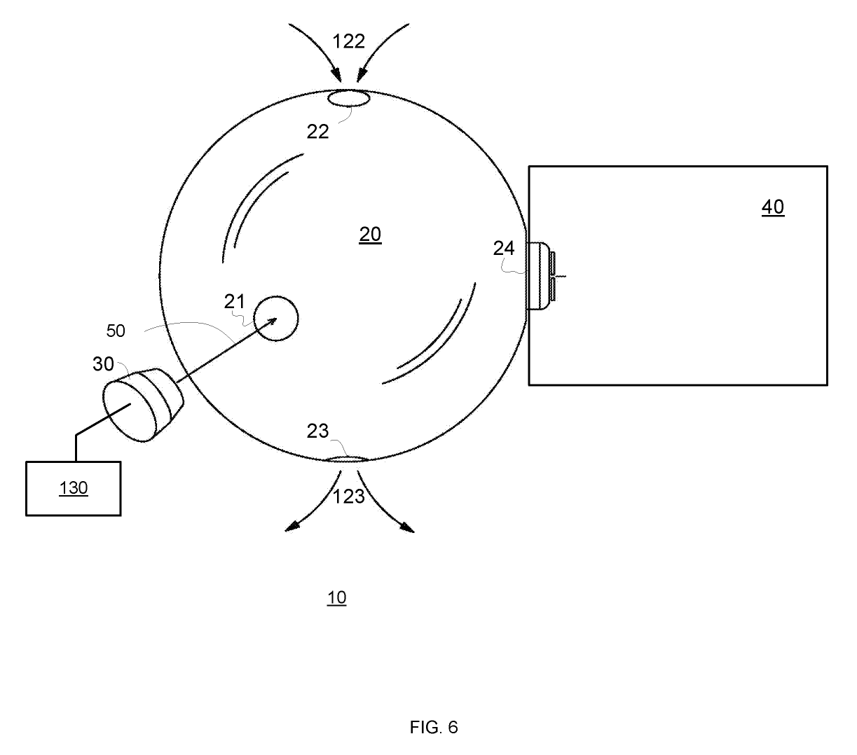

FIG. 6 illustrates a movement unit (such as a mechanical stage) 130 that introduces movement between the sphere 20 and the light source 30. The movement may change the optical axis of the light source and therefore may change the number of scattering of the light beam within the sphere.

There may be provided a method for sensing gas using the mentioned above sensor.

The method may include:
a. Directing a beam of light through a first opening of a sphere.
b. Detecting a light beam that exits the sphere through a second aperture.
c. Analyzing the light beam that exited by a spectrometer to find absorbance pattern of Hydrogen Sulfite. The light beam enters the first aperture while propagating along an optical axis. The intersection point of the optical axis and the sphere (at a point that is opposite to the first aperture) is spaced apart from the second aperture. In other words—the light beam has to be reflected and/or scattered from the interior of the sphere before exiting through the second aperture.

The positions, shapes and sized of the first and second gas apertures and/or of the first and second aperture may change from those illustrated in FIGS. 1 and 2.

FIG. 5 illustrates an example of method 400.

Method 400 may start by step 410 of directing, by a light source of the gas sensor, a light beam through a first aperture of a sphere of the gas sensor.

The sphere may also include a second aperture and a first gas opening.

The sphere may also include a second gas opening.

The Hydrogen Sulfite may flow within the sphere and between the first and second gas apertures.

An imaginary arc may pass through the first gas opening, a second gas opening, and the first aperture, wherein the first aperture is located at the center of the imaginary arc and the first and second gas openings are located at opposite ends of the imaginary arc.

The wavelength of the light beam may range between 180 and 300 nanometer.

The first aperture may have a circular shape and the second aperture may be a slit.

Step 410 may be followed by step 420 of receiving, by the first aperture, the light beam.

Step 420 may be followed by step 430 of scattering multiple times, by an interior of the sphere, the light beam to provide a scattered light beam that exits the sphere through the second aperture.

The multiple times may exceed two, may exceed three and the like.

The repetition defines a folded optical path that may exceed a diameter of the sphere—for example by a factor that ranges between 2.5 and five. The factor may exceed one and may even exceed five.

When the light is scattered N times during step 430—then N-1 sets of scattered light beams are generated. A set of scattered beams is generated during each scattering event. In order to enable the N scattering—the second aperture is positioned outside a propagation path of each of the N-1 sets of scattered light beams.

Step 430 may be followed by step 440 of receiving, by a gas analyzer of the gas sensor, the scattered light beam from the second aperture.

Step 440 may be followed by step 450 of analyzing the scattered light beam by searching for one or more signs that are indicative of a presence of the Hydrogen Sulfite in the sphere.

The one or more signs may be absorbance patters of the Hydrogen Sulfite.

Step 430 and/or step 440 may be followed by step 410.

The light beam may be a continuous light beam or a pulsed light beam.

The gas sensor may be static during step 430.

Alternatively—method 400 may include step 402 of moving the gas sensor in relation to its environment—thereby scanning a presence of the Hydrogen Sulfite within a certain region of interest. The method may include introducing movement between the sphere and the light source.

The Hydrogen Sulfite may flow into and/or out of the sphere in a forced manner.

Method 400 may include step 404 of forcing the Hydrogen Sulfite to move through the sphere.

For example—step 404 may include inducing the Hydrogen Sulfite to flow from the first gas aperture and to exit the sphere through a second gas aperture (or even back through the first gas aperture).

Method 400 may include generating an alert or any notification about the results of the analysis. The alert or notification may be wirelessly transmitted and/or transmitted by wire, and/or stored in a memory unit and/or outputted by a man machine interface. The alert may be an audio-visual alert. The alert or notification may be short-range and/or long-range transmitted.

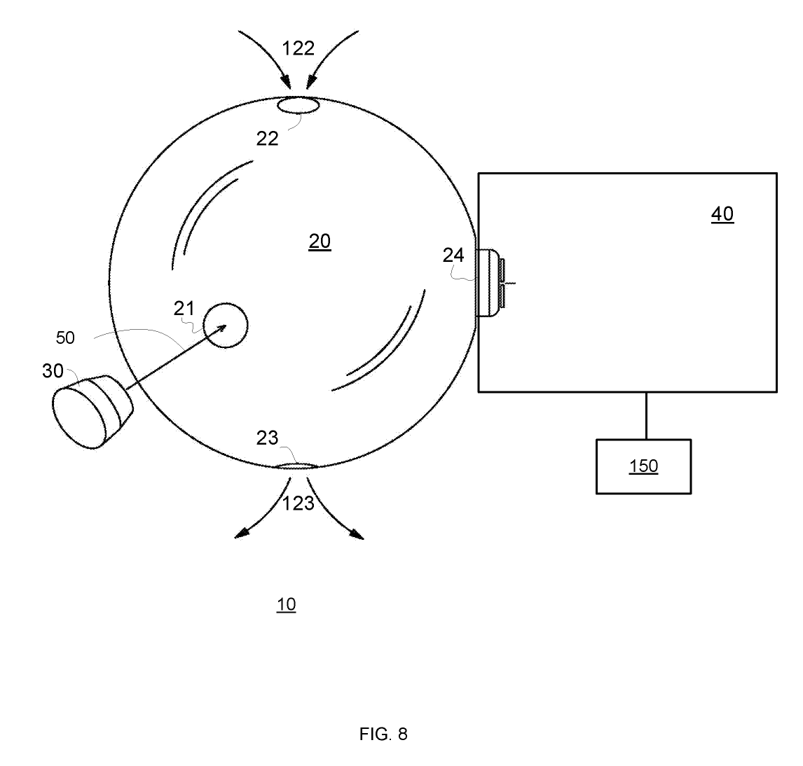

FIG. 8 illustrates unit 150 that is coupled to gas analyzer 40.

Unit 150 may include at least one out of:
a. A processor for processing the analyzer result and/or generate the indication and/or alert, (the processing may be done by the analyzer itself).
b. A memory unit for storing the alert and/or the indication.
c. A man machine interface for outputting an alert and/or a notification.
d. A communication module.

In the foregoing specification, the invention has been described with reference to specific examples of embodiments of the invention. It will, however, be evident that various modifications and changes may be made therein without departing from the broader spirit and scope of the invention as set forth in the appended claims.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

The terms “including”, “comprising”, “having”, “consisting” and “consisting essentially of” are used in an interchangeable manner. For example- any method may include at least the steps included in the figures and/or in the specification, only the steps included in the figures and/or the specification. The same applies to the pool cleaning robot and the mobile computer.

We claim:

1. A gas sensor for sensing Hydrogen Sulfite, wherein the gas sensor comprises:
a sphere; and
a gas analyzer;
wherein the sphere comprises a first aperture, a second aperture, and a first gas opening;
wherein an interior of the sphere is configured to scatter multiple times, a light beam that enters the sphere through the first aperture, before a scattered light beam exits the sphere through the second aperture;
wherein the gas analyzer is configured to receive the scattered light beam from the second aperture, and to analyze the scattered light beam by searching for one or more signs that are indicative of a presence of the Hydrogen Sulfite in the sphere.

2. The gas sensor according to claim 1 further comprising a light source that is configured to direct the light beam through the first aperture.

3. The gas sensor according to claim 1 wherein the multiple times exceed three.

4. The gas sensor according to claim 1 further comprising a gas flow control unit that is configured to force the Hydrogen Sulfite to flow through the sphere.

5. The gas sensor according to claim 1 wherein the one or more signs are one or more absorbance patterns that are indicative of an absorbance of the Hydrogen Sulfite in the scattered light beam.

6. The gas sensor according to claim 1 wherein the light beam is configured to be scattered N times before exiting from the second aperture, wherein the scattering generates N-1 sets of scattered light beams, wherein the second aperture is positioned outside a propagation path of each of the N-1 sets of scattered light beams.

7. The gas sensor according to claim 1 wherein the interior of the sphere is configured to scatter the light beam multiple times thereby defining a folded optical path that exceeds a diameter of the sphere by a factor that ranges between 2.5 and five.

8. The gas sensor according to claim 1 wherein the interior of the sphere is configured to scatter the light beam multiple times thereby defining a folded optical path that exceeds a diameter of the sphere by a factor that exceeds two.

9. The gas sensor according to claim 1 wherein a wavelength of the light beam ranges between 180 and 300 nanometer.

10. The gas sensor according to claim 1 wherein the first aperture has a circular shape and the second aperture is a slit.

11. The gas sensor according to claim 1 wherein the sphere further comprises a second gas opening.

12. The gas sensor according to claim 11 wherein an imaginary arc passes through the first gas opening, a second gas opening, and the first aperture, wherein the first aperture is located at the center of the imaginary arc and the first and second gas openings are located at opposite ends of the imaginary arc.

13. The gas sensor according to claim 1 further comprising a movement unit that is configured to move a light source of the gas sensor, the sphere and the gas analyzer.

14. The gas sensor according to claim 1 further comprising a movement unit that is configured to introduce movement between the light source and the sphere.

15. A method for sensing Hydrogen Sulfite by a gas sensor, the method comprises:
receiving, by first aperture of a sphere of the gas sensor, a light beam; wherein the sphere further comprises a second aperture and a first gas opening;
scattering multiple times, by an interior of the sphere, the light beam to provide a scattered light beam that exits the sphere through the second aperture;
receiving, by a gas analyzer of the gas sensor, the scattered light beam from the second aperture; and
analyzing the scattered light beam by searching for one or more signs that are indicative of a presence of the Hydrogen Sulfite in the sphere.

16. The method according to claim 15 further comprising directing by a light source of the gas sensor, the light beam through the first aperture.

17. The method according to claim 15 wherein the multiple times exceed three.

18. The method according to claim 15 further comprising forcing, by a gas flow control unit, the Hydrogen Sulfite to flow through the sphere.

19. The method according to claim 15 wherein the one or more signs are one or more absorbance patterns that are indicative of an absorbance of the Hydrogen Sulfite in the scattered light beam.

20. The method according to claim 15 comprising scattering the light beam, by the sphere, N times before exiting from the second aperture, thereby generating N-1 sets of scattered light beams, wherein the second aperture is positioned outside a propagation path of each of the N-1 sets of scattered light beams.

21. (canceled)

22. (canceled)

23. (canceled)

24. (canceled)

25. (canceled)

26. (canceled)

27. (canceled)

28. (canceled)
